(19) United States
(12) Patent Application Publication
(10) Pub. No.: US 2003/0134265 A1
(43) Pub. Date: Jul. 17, 2003

(54) Screening method for nucleic acids

(76) Inventors: Peter Buckel, Bernried (DE); Ulrich Pessara, Weilheim (DE); Wolfgang Liebetrau, Penzberg (DE); Dieter Link, Martinsried (DE)

Correspondence Address: Arent Fox Kintner, Plotkin & Kahn, Suite 600, 1050C Connecticut Avenue NW, Washington, DC, 20036-5339, US

(21) Appl. No.: 10/168,683

(22) PCT Filed: Dec. 21, 2000
(86) PCT No.: PCT/EP00/13132

(30) Foreign Application Priority Data
Dec. 23, 1999 (DE) 199 62 604.9
Jul. 2, 2000 (DE) 100 36 175.7

(51) Int. Cl. C12Q 1/00; C12Q 1/68; C12N 15/85

(52) U.S. Cl. 435/4; 435/6; 435/455

(57) ABSTRACT

The invention relates to a method for determining the activity of nucleic acid sequences and to the use of the nucleic acid sequences identified in this way for providing diagnostic and therapeutic agents.

The invention relates to a method for determining the activity of nucleic acid sequences and to the use of the nucleic acid sequences identified in this way for providing diagnostic and therapeutic agents.

Previous methods for identifying the activity of nucleic acid sequences include expression cloning which generally comprises introducing a gene library, i.e. a multiplicity of different nucleic acid sequences together into a population of target organisms by transfection or transformation. After expression of the nucleic acid transfected or transformed, individual target organisms having a sought-after activity, for example expression of an antigen for an antibody etc., are physically separated from the remaining target organisms. The DNA transfected or transformed which is frequently present in the form of an extrachromosomal plasmid is isolated from the individual target organisms isolated in said manner and is amplified, for example by propagation in bacteria cells. The DNA is then again isolated from said bacteria cells and transfected into mammalian cells. Several rounds of this procedure increase the amount of the sought-after nucleic acid. Said methods have the disadvantages that (a) one cell can take up several genes (mixing of activities, loss of a meaningful result, reduction in the specific signal, etc.); (b) only a few cells show the desired effect (no statistical interpretations possible); (c) automation is possible only with difficulty, since a linear process is absent (circulating mixtures); (d) DNA must remain intact in the cells for subsequent isolation (not workable, for example, for apoptosis).

The German patent application 199 50 585.0 proposes a method for identifying nucleic acid sequences having a non-selectable activity, in particular apoptosis activity, in a target cell, in which method a DNA library is provided in host cells which contain an expression vector operatively linked to an expression control sequence active in a target cell, the host cells are cultured, the expression vector is obtained from the cultured host cells, the expression vector and a reporter vector are introduced into a target cell and the reporter vector activity in the target cells or in the supernatant of their cultures is determined as a qualitative or quantitative measure for the non-selectable activity of the nucleic acid to be studied.

The present invention relates to a method for determining the activity of nucleic acid sequences, which also includes nucleic acid sequences having a selectable activity.

The method comprises the steps:

(a) parallel introduction of a multiplicity of expression vectors containing in each case a nucleic acid sequence to be studied operatively linked to an expression control sequence into a multiplicity of in each case separate populations of target organisms of the same type, where in each case only a single nucleic acid sequence or a small number of various nucleic acid sequences are introduced into a separate population of target organisms of the same type,

(b) effecting of expression of the nucleic acid sequence in the target organisms, and

(c) determination of the activity of the nucleic acid sequence in the individual populations of target organisms.

The method is a screening procedure for parallel determination of a multiplicity of nucleic acids. Owing to the introduction of a single nucleic acid sequence or a small number of nucleic acid sequences into in each case separate populations of target organisms, populations of target organisms treated in the same way are formed, which allow for sensitive evaluation of the screening. The fate of the nucleic acid sequences introduced into the target organism is irrelevant here, since preferably only one aliquot of the nucleic acid sequences to be studied is used and the remainder is retained for subsequent studies.

The nucleic acid sequences to be studied can in principle come from any sources, for example from eukaryotes such as plants, vertebrates, e.g. mammals, fungi, parasites, etc., or else bacteria, archaebacteria or viruses or from synthetic or semisynthetic sources. They are selected, for example, from genomic sequences, cDNA sequences, cDNA fragments or part sequences or else from synthetically generated sequences such as, for example, antisense molecules or combinatorially modified nucleic acid sequences of any origin or sequences suitable for RNAi.

The method of the invention makes it possible to determine nucleic acid sequences having any activity, as long as said activity can be determined in the target organisms transfected or transformed with the nucleic acid sequences. The activity of the nucleic acid sequences can be a selectable or non-selectable activity. A non-selectable activity in this connection means that the relevant nucleic acid cannot stably be (over)expressed in recombinant form in a target organism, for example because it inhibits cell growth or leads to cell death. On the other hand, it is of course also possible to determine nucleic acid sequences having a selectable activity; however, it has to be pointed out that the method of the invention includes no selection, but a serial study on a multiplicity of transfected or transformed target organisms. The selectability expresses only the theoretical possibility of being able to use the appropriate cellular effect also in a selection plan for isolating nucleic acids. The use of a screening is advantageous, since this overcomes the abovedescribed disadvantages of positive selection.

Preferred examples of the activity of nucleic acid sequences are DNA repair, transcriptional activation of genes, activation or inhibition of protein secretion or protease activity, activation or inhibition of telomerase activity, generation or elimination of protein/protein or protein/DNA interactions, etc.

Step (a) of the method comprises providing a multiplicity of expression vectors containing in each case a nucleic acid sequence to be studied operatively linked to an expression control sequence and introducing said multiplicity of expression vectors in each case separately into a multiplicity of populations of target organisms of the same type. The multiplicity of expression vectors may be a DNA library.

Said DNA library is preferably, in particular in the case of a cDNA library, a normalized library, i.e. a library with reduced content of abundant species. The preparation of such normalized libraries has been described by Sasaki et al. (Nucleic Acids Res. 22 (1994), 9987-9992). Reduction in the abundant species content in a population of mRNA molecules is achieved by addition of, for example, cDNA molecules immobilized on latex beads, where appropriate in several hybridization cycles. However, it is also possible to use non-normalized DNA libraries as starting material, for example libraries containing a collection of specific nucleic acids such as genes.

In this context, it is possible, after determination of substantial parts of or complete genomic sequences or of all expressed sequences of an organism, to use gene libraries which include only a single copy of each gene. Instead of reducing the abundant gene content the collection of all transcribed sequences is used as a starting point here and the library is assembled accordingly. At the moment, gene libraries of this kind are being constructed and should contain each gene of a species in an expressible form (Strausberg et al., Science 286 (1999), 455-457).

The nucleic acid sequences to be studied are located in an expression vector which is active in the target organism desired in each case, preferably a eukaryotic cell or a eukaryotic organism, and in particular a mammalian cell, i.e. the nucleic acid to be studied is operatively linked on the expression vector to an expression control sequence constitutively or controllably active in the target organism. Since a selection of the expression vector in the target organism need not be carried out, the presence of elements allowing selection in the target organism is not necessary. In some embodiments, the missing of elements allowing selection in the target organism is in fact preferred.

Expediently, the expression vector is an extrachromosomal vector and in particular a transiently transfectable plasmid. Alternatively, however, a stable episomal expression vector may also be employed. Expression vectors of this kind are known to the worker skilled in the field of molecular biology and are described, for example, in Sambrook et al., Molecular Cloning. A Laboratory Manual (1989), Cold Spring Harbor Laboratory Press, or in other standard text books.

The expression vectors can be generated in any manner, for example by culturing in host cells which are preferably bacteria cells, in particular Gram-negative bacteria and particularly preferably E. coli cells. In this case, the expression vector expediently contains elements which make replication and selection in the host cell possible. The host cells are preferably cultured individually, for example by plating out single clones of a nucleic acid library onto solid culture plates or appropriate dilution of liquid culture media. Optionally it is also possible to culture a plurality of host cells, for example small pools of not more than ten different clones, together, although this is less preferred in most cases.

In order to obtain DNA from host cells, methods known from the prior art can be used for isolating extrachromosomal DNA (see, for example, Sambrook et al., supra). However, in this connection care must be taken that the DNA isolated from the host cell should be sufficiently pure in order to allow subsequent transfection of the target organism with high efficiency. For bacterial host cells, an alkaline lysis is preferably carried out. The quality of the plasmid DNA obtained may be improved by adsorption to a solid matrix, in particular a silica adsorption matrix, washing with organic solvents and subsequent elution. The expression vectors may be introduced into the cells in circular or linear form.

Alternatively, the expression vectors may also be generated in sufficient quantity by in-vitro amplification, for example by polymerase chain reaction (PCR), ligase chain reaction (LCR) or rolling circle amplification.

Furthermore, step (a) includes introducing an expression vector into a population of target organisms of the same type, with preferably in each case only a single expression vector being introduced into such a population of target organisms. Preferred target organisms used are eukaryotic cells such as, for example, mammalian cells, in particular human cells, but also fungi such as yeasts, parasites such as trypanosomes, etc. The use of intact organisms is advantageous compared with cell cultures, since many diseases and cellular effects can be observed only through the interaction of many different cell types in a higher organism. It is also possible to use target organisms of prokaryotic origin, for example bacterial cells. From among prokaryotic organisms, pathogens whose physiology can be studied by said screening are preferably used, especially in relation to therapeutic measures. For this purpose, it is possible to study, for example, individual steps of the propagation cycle, such as the formation of surface structures for adhesion and penetration into the host cell. Using said screening, it is possible to isolate nucleic acids for proteins which act inhibitorily on the manifestation of the particular cellular effect of the (pathogenic) organisms. This is especially advantageous when studying processes in cells which are under no great selection pressure, since said processes are not essential for survival of said cells (e.g. adhesion receptors). Likewise, it is also possible to use more complex organisms as target organism, with the exception of humans, for example zebrafish, mice, Drosophila or nematodes such as C. elegans. The source of the nucleic acids to be studied and the target organism may be identical or different, for example different mammalian species.

The target organisms may have been mutated or genetically manipulated, where appropriate, i.e. they can, compared with a corresponding wild type organism, overexpress one or more nucleic acid sequences, where appropriate nucleic acid sequences from foreign organisms. Alternatively, it is also possible to use target organisms in which, compared with the wild-type, a nucleic acid sequence is expressed only at reduced levels, only at low levels, not at all or in mutated form. Yeast, C. elegans and Drosophila have long been established as model organisms in genetics. It is therefore possible to access many mutations which can be employed in the screening (Hartwell et al., Science 248 (1997), 1064-1068). Said mutations correspond partially to mutations which have been found to be causing human diseases. Alternatively, such mutations can be generated in said model organisms. Said genetic defects can be repaired by introducing complementing nucleic acids using the screening described herein. This may lead to a quick identification of the genes responsible for particular mutations. On the other hand, it is also possible to isolate nucleic acids which correct the original defects, without being the originally mutated gene itself (“compensation”). The use of said screening is advantageous, since deregulated expression of many genes, and thus also of those which can complement mutations, is often lethal for organisms in later developmental stages and can therefore not be isolated by conventional positive selection.

For several organisms, “RNA interference (RNAi)” has been described. Here, the endogenous gene is inactivated by expression of double-stranded RNA (Bosher and Labouesse, Nat. Cell. Biol. 2 (2000), E31-36). The screening described here is particularly well suitable for genetically detecting genes whose inactivation leads to detectable changes. This may be carried out both in intact organisms and in cell lines. Organisms and cell lines which have been genetically manipulated such that RNAi is possible are also suitable. C. elegans is particularly suitable for such studies, since the appropriate gene sequences can only be expressed in this organism by feeding of bacteria containing the clones from a gene library. This takes place evidently by uptake of the bacteria in the gut of the animals and by the disruption and by the release of the DNA in C. elegans (Timmons and Fire, Nature 395 (1998), 854). RNAi requires double-stranded RNA. In the screening proposed here, however, only sense RNA is generated by the particular plasmid. In order to adopt RNAi for said screening, it is therefore necessary to use plasmids which contain the cDNAs with two flanking (5′ and 3′) promoters. This should lead to the formation of statistically equal amounts of sense and antisense RNA, which should knock out the corresponding endogenous gene via RNAi. The read-out can then be achieved by digital high-throughput imaging systems such as, for example, the Becton Dickinson HTS cell imaging system.

In particular cases, the use of the target organisms containing a reporter vector is preferred. Alternatively, it is also possible to introduce a reporter vector together with the expression vector into the target organism.

The expression vector and, where appropriate, reporter vector, are introduced into the target organisms according to methods for (co)transfection, (co)transformation or (co)infection of cells, which are in principle known from the prior art. However, a method should be chosen which allows high efficiency in introducing foreign nucleic acids into the target organism, for example an efficiency of at least 1%.

Eukaryotic target cells are transfected or cotransfected preferably by calcium phosphate coprecipitation, lipofection, electroporation, particle bombardment, use of bacterial proteins or viral infection (retroviruses, adenoviruses, sendai viruses, etc.) In the target cells generated by transfection or cotransfection, the expression vector may be expressed transiently and the reporter vector may be expressed transiently or stably. Transient expression is preferred.

The preferred cotransfection advantageously uses the expression vector in a molar excess based on the reporter vector. The molar ratio between reporter vector and expression vector is particularly preferably 1:2 to 1:20. When using the expression vector in molar excess, the presence of the reporter vector in the target organism may serve as a marker for the simultaneous presence of the expression vector in the target organism, since during a cotransfection organisms take up the plasmids employed according to the molar ratio of said plasmids in the cotransfection mixture.

The reporter vector is preferably chosen in a way such that there is no immediate functional connection between reporter and expression vector, i.e. that a gene product encoded by the expression vector does not directly act on the activity of the reporter vector, but that a gene product encoded by the expression vector acts only indirectly, i.e. via influencing the metabolism of the target organism, on the activity of the reporter vector. However, embodiments of the screening, which allow detection of direct interactions between reporter vector and expression vector, are also possible.

The reporter vector which has been cotransfected together with the expression vector or is already present in the target organism generally contains a nucleic acid sequence expressible in the target organism, which codes for a detectable gene product. The reporter vector is preferably an extrachromosomal vector, particularly preferably a transiently transfectable plasmid. On the other hand, it is also possible to use a stable episomal or chromosomal reporter vector. In this case, the reporter vector contains elements which make selection and, where appropriate, replication in the target organism possible. The detectable gene product may be expressed via a constitutive or controllable expression control sequence, preferably via a constitutive expression control sequence.

In a particularly preferred embodiment, the gene product encoded by the reporter vector is a secreted enzyme, i.e. an enzyme which is secreted by the target organism. Examples of such enzymes are secreted alkaline phosphatase (SEAP) (Berger et al., Gene 66 (1988), 1-10) and luciferase (Lui et al., Gene 202 (1977), 141-148). Particular preference is given to using SEAP as secreted enzyme. When using secreted gene products as reporter system, the activity is determined in the supernatant of target cell cultures. On the other hand, the detectable gene product encoded by the reporter vector may also be a non-secreted polypeptide which is intracellularly detectable in an intact cell, for example a fluorescent protein such as GFP. Likewise, the detectable gene product expressed by the reporter vector may also be a membrane-bound polypeptide detectable, for example, by incubation with antibodies or affinity ligands.

Incidentally, the method of the invention also allows the use of a plurality of reporter vectors of which one is already present (chromosomally or extrachromosomally) in the target organism and another one is introduced into the target organism together with the expression vector by means of cotransfection. This avoids having to use too much DNA as reporter plasmid and not enough DNA of the genes to be tested from the library. Thus it is possible, for example, for the cotransfected plasmid to code for a nuclear GFP variant which is used in order to identify the transfected cells. The cells already stably express a fusion protein containing another spectral GFP variant which is located in the cytoplasm. Appropriate GFP variants have been described in the relevant literature (Haseloff, Meth. Cell. Biol. 58 (1999), 139-151). If one of the genes from the library leads to translocation of the fusion protein into the nucleus, then this can be detected by the overlap of the different emissions of the two GFP variants by appropriate imaging programs.

Moreover, it is possible to use cells into which reporter plasmids are stably integrated at specific positions in the genome. In such knock-in experiments, an endogenous gene is replaced by the reporter plasmid (Elefanty et al., PNAS USA 95 (1998), 11897-11902) whose activity then reflects the transcriptional activation of the replaced gene. Stably transfected cells may also be used for a telomerase activity assay. For this purpose, a reporter plasmid can stably be integrated into the telomeres. If the telomere length is reduced, the activity of the reporter plasmid, too, should be reduced or (in the case of only one copy) completely reduced. It is further possible to use cell lines expressing GFP fusion proteins of secreted proteins. Since the proteins are secreted, no fluorescence is observed in the cells. If the cells are transfected with genes leading to inhibition of the secretion, an increase in GFP fluorescence is observed. This has already been shown by the physical inhibition of the transport of a GFP fusion protein (Kaether and Gerdes, FEBS Lett. 369 (1995), 267-271). Several diseases are based on an increased protein secretion (e.g. Alzheimer's disease). Conversely, it is possible to identify genes leading to maturation of secreted proteins: the activity of the protein may then be detected in the medium by suitable methods. This may involve fusion proteins having catalytic properties. Alternatively, it is also possible to determine the decrease in the fluorescence of GFP fusion proteins in the cytoplasm of the cells.

Step (b) of the method of the invention includes expression of the nucleic acid sequence in the target organisms. For this purpose, target organisms, depending on the expression vectors used in each case, are cultured under suitable conditions which allow expression of the nucleic acid sequence to be assayed which is operatively linked to the expression control sequence of the expression vector. A particularly good expression efficiency can be achieved by the preferred expression of only a single type of nucleic acid per population of target organisms.

Step (c) of the method includes determining the activity of the nucleic acid sequence in the individual populations of target organisms. The determination includes in each case a separate study of individual target organism populations, preferably of in each case a plurality of target organisms and particularly preferably of essentially all target organisms of the total population. The size of the total population depends on the particular target organisms. If the target organisms are cells, the population size is preferably from about 10² to 10⁶ for eukaryotic cells and preferably from about 10⁵ to 10¹⁰ for prokaryotic cells. In more complex target organisms, the population size is of course smaller and should be about 2 to 10⁴.

Determination of the biological activity of the nucleic acid sequence may in principle include determination of any phenotypically detectable effects, for example morphological changes, changed growth behavior, etc. If the target organism contains a reporter vector, changes in the activity of the reporter vector, i.e. in particular of the gene product encoded by the reporter vector, can be used as a measure for the activity of the nucleic acid sequence to be studied. Said determination method is based on the fact that the nucleic acid sequence to be studied, located on the expression vector, influences in the target organism, after expression, the cell metabolism which in turn in a measurable way influences the activity of the reporter vector or of the detectable gene product encoded thereby. It is possible here to determine the activity of the reporter vector at at least two points after transfection or cotransfection, with the first point in time being chosen such that expression of the nucleic acid sequence contained on the expression vector does not yet influence the activity of the reporter vector and thus being able to serve to establish a base activity for the target organism studied in the particular case. The second point in time is chosen such that expression of the nucleic acid sequence contained on the expression vector already has a measurable influence on the activity of the reporter vector, as long as the nucleic acid sequence present in the particular organism has the activity studied. In this way it is possible to determine, independent of the base activity of the reporter vector, which activity depends on the transfection efficiency of the transfection or cotransfection, the activity of a nucleic acid sequence introduced into a particular organism.

If the method of the invention is used for identifying nucleic acid sequences influencing secretory properties of the target cell, measuring the activity of the reporter vector in the supernatant of the cells is sufficient for identifying the desired nucleic acid sequences. In other embodiments of the method, the detectable gene product encoded by the reporter vector, for example a fluorescent protein such as GFP, can be expressed intracellularly to mark the transfected organisms. This marking may be combined, where appropriate, with the detection of additional cellular parameters, for example detection of surface markers with antibodies or receptor ligands. These additional parameters can be detected by using fluorescent reagents. The presence, subcellular distribution or/and intensity of the marking(s) on the organisms can then be determined by fluorescence cytometry, for example by means of FACS (fluorescence activated cell sorting) or imaging assays.

The method may be carried out as at least partially automated serial screening, in which preferably steps (a) to (c) are carried out in each case in parallel on at least 50 populations of target organisms.

In another embodiment of the invention, it is possible to identify genes which, due to overexpression, have a specific influence on the cell cycle of the target cells. For example, overexpression of the gene for the p21 protein shows such an effect on arresting the G1 phase (Gartel et al., Proc. Soc. Exp. Biol. Med. 213 (1996), 138-149).

In a preferred embodiment, the method of the invention is carried out as multi-stage protocol. In this connection, in particular nucleic acid sequences which can prevent or inhibit, respectivly, or enhance or cause, respectively, the manifestation of a particular physiological reaction of the target organism are determined. For this purpose, a stimulus, for example a pharmacon or therapeutic active substance, whose activity can be influenced by the nucleic acid to be studied is added to the target organisms. The stimulus which may be one or more physiologically active substances, for example receptor ligands, medicaments, cytokines, etc., may be added before, during or after introducing the nucleic acid sequence to be studied in the population of target organisms. Thus, the method of the invention may additionally represent a screening for determining interactions between the biological activities of nucleic acids to be studied and stimuli.

The method described herein therefore can serve to optimize therapeutic reagents. Thus it is possible to determine genes which can inhibit the effect of the therapeutic agent. If said genes at a later stage are inhibited by an appropriate action when adding the therapeutic agent, the effect of said therapeutic agent can be enhanced.

For example, the inducible transcription factor NF-κB can be inhibited by sodium salicylic acid (aspirin) (Kopp and Ghosh, Science 265 (1994), 956-959). This is achieved by inhibiting a kinase which can phosphorylate the specific inhibitor IκB and thus leads to its degradation (Yin et al., Nature 396 (1998), 77-80). It should be possible to remove inhibition by aspirin by transfection with and subsequent overexpression of the transactivating subunit p65 (relA). As a result, a gene is identified which is not a direct target of the therapeutic agent and whose inhibition can enhance the effect of aspirin.

The screening can also be used to determine genes which enhance the activity of pharmaceuticals, for example cotransfection of IκB can enhance inhibition of the inducible transcription factor NF-κB by aspirin. Here too, the identifiable gene is not the one upon which the pharmacon itself acts.

Both enhancing and inhibiting the effect of a pharmacon make it possible to determine novel approaches for therapeutic interventions in the cell.

In yet another embodiment of the invention, it is possible to culture the population of target organisms, after introducing the nucleic acid sequence to be studied, together with further organisms (test organisms), for example a further cell line, and to study the reaction of the test organisms to the nucleic acid sequence to be studied. The test organism may contain a reporter vector as described above. In this way it is possible to detect not only secreted proteins but also those proteins which are membrane-bound and which can act via direct cell/cell interactions, where appropriate in combination with surface markers of the test organism.

The target organisms may be infected with viruses before, during or after introducing the nucleic acid sequence to be studied and the influence of the nucleic acid sequence to be studied on the behavior of the target organisms toward the viruses, for example inhibition or activation of viral replication or penetration of the virus into the cells, can be determined.

Instead of viruses, however, it is also possible to use other pathogenic organisms such as, for example bacteria. In contrast to the above-described application of the screening, here the effect of the changes caused by the transfected genes in the target organisms on the propagation cycle of the host organisms (viruses, bacteria) is studied and not the direct effect on the target organisms. For this application, it can be expected, for example, to isolate genes which downregulate receptors for the penetration of the viruses into the cells.

Thus, it is possible, for example, to add to the transfected cells another cell line containing a reporter plasmid which has a DNA binding site for a specific transcription factor (e.g. NF-κB) and therefore is activated by said factor.

In this way, the screening can detect secreted or membrane-bound proteins which lead to activation of a transcription factor. For example, the inducible transcription factor NF-κB (Baeuerle and Baltimore, Cell 87 (1996), 13-20) can be detected in the test organisms, after genes for activator proteins such as, for example, TNF (Baldwin, Annu. Rev. Immunol. 14 (1996), 649-683) have been transfected into the cells.

Optimization of the biological activity of nucleic acid sequences represents yet another embodiment of the invention. For this purpose, a given nucleic acid sequence may be subjected to a mutagenesis, for example a mutagenesis by PCR, by random PCR (Cadwell and Joyce, PCR Methods Appl. 2 (1992), 28-33), by DNA shuffling (Harayama, Trends Biotechnol. 16, 1998, 76-82) or to a fusion with statistical nucleic acid sequences (at the ends or within the given nucleic acid sequence) in order to provide the multiplicity of nucleic acid sequences required for step (a) of the method. Preferred examples of the screening of nucleic acid interactions mutated in this way are nucleic acids coding for a receptor, which are to be tested for optimized interaction with one or more ligands. Conversely, it is possible to appropriately optimize nucleic acid sequences coding for a peptide ligand or protein ligand capable of binding to a receptor, whose interaction with the receptor is to be optimized. Appropriate experiments may be carried out by mutagenesis of cytokines by means of DNA shuffling to optimize their effect (Chang et al., Nat. Biotechnol. 17 (1999), 793-797).

Yet another example is mutagenesis of nucleic acid sequences coding for antigen binding regions of antibodies, for example of single-chain antibodies, in order to optimize the ability of the gene products encoded by the sequences to bind to particular antigens. Yet another example is mutagenesis of effector sequences, for example the TAT sequence which promotes uptake of proteins into cells. For this purpose, the TAT sequence can be fused to a particular gene and to determine the TAT variant optimal for the particular gene product by mutagenesis of the TAT sequence. In this embodiment, the screening is preferably carried out not on unknown nucleic acid sequences, but on variants of known nucleic acid sequences, which have been generated by mutagenesis or natural mutation. It is possible to carry out said nucleic acid optimization over several cycles, with nucleic acid sequences which have a desired biological activity and have been identified in a first cycle being subjected to further mutagenesis procedures in one or more subsequent cycles.

The genes identified by the method of the invention can be used for providing diagnostic and therapeutic agents. Thus it is possible to use different target cells (normal cells, tumor cells) when carrying out the method. It is also possible to utilize for screening cells having specific genetic modifications such as, for example, cells having activated oncogenes or inactivated tumor suppressor genes, and also virus-infected cells. As a result, it is possible to identify or isolate nucleic acid sequences which are selectively active in particular cell types, for example genes which act selectively in tumor cells or virus-infected cells. It would then be possible, for example, to express said genes in patients to control tumors or viral infections by gene therapy.

The method of the invention can be applied universally to any types of target organisms. Thus it is possible, when using bacterial cells as target organisms, to identify nucleic acid sequences coding for an antibiotics resistance protein or for a protein with antibacterial action. “Defensins” have been described for various organisms (Hancock and Lehrer, Trends Biotechnol. 16 (1998), 82-88). These peptides are preferably toxic for bacteria, since they lead to lysis of the bacterial membrane.

When using patient cells having a genetic defect as target organisms, it is possible to screen for nucleic acid sequences which are capable of repairing said defect. Furthermore, the screening method can be extended to optimizing transfection for application in gene therapy and identification of reporter plasmids which can detect kinase activation.

Gene therapy is optimized by transfecting the cells with genes of a gene library in order to determine the nucleic acids leading to improved uptake of vectors utilized in gene therapy.

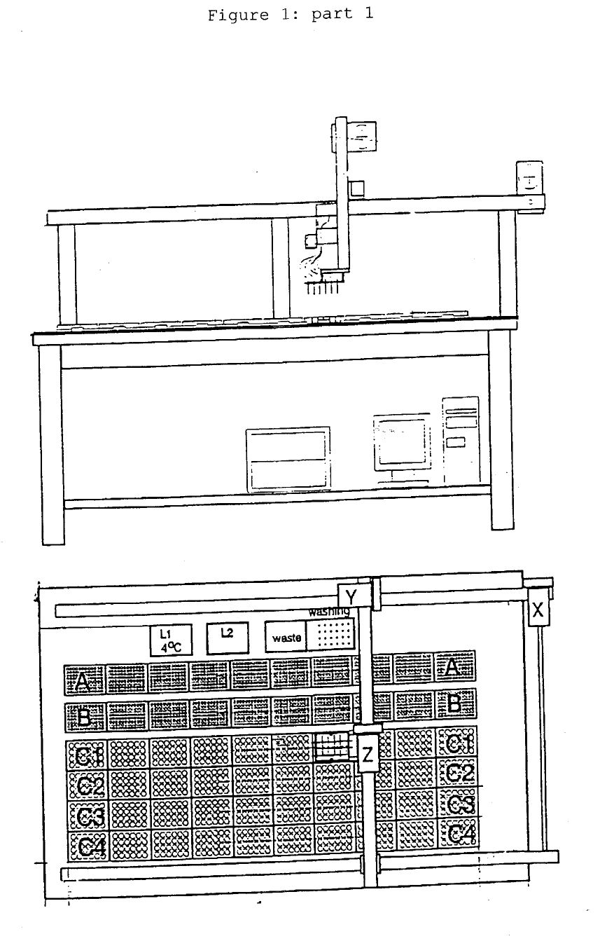

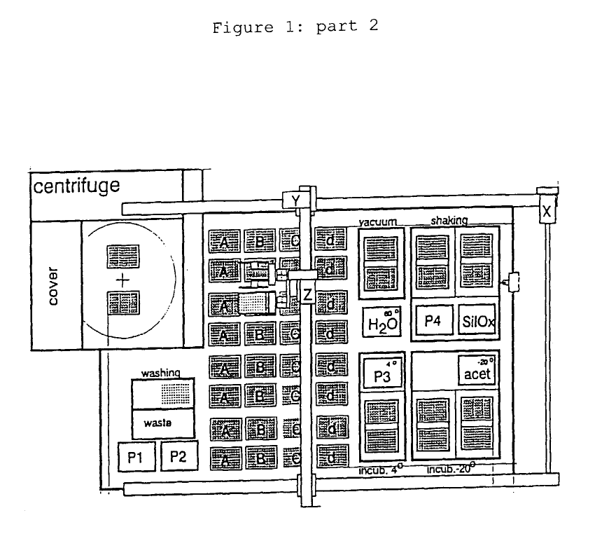

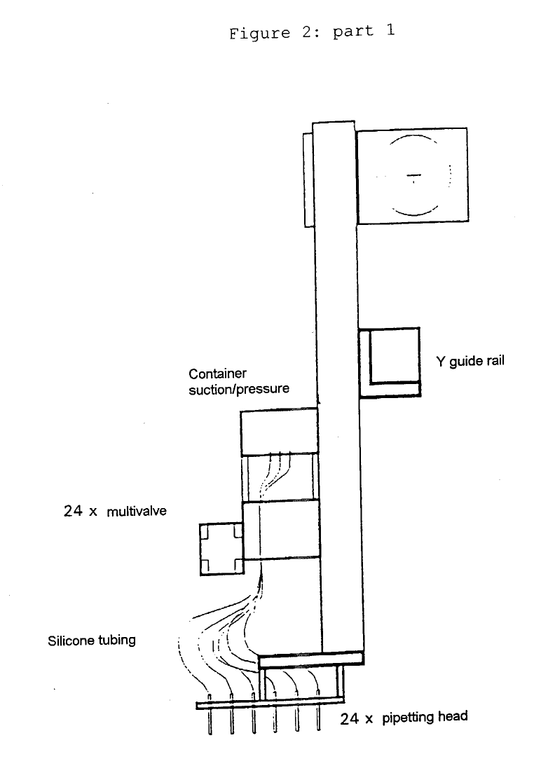

The method can be automated, for example, in an apparatus according to DE 199 50 585.0. Said apparatus preferably contains two robots: one for the isolation of plasmid DNA from the host cells and one for transfection of target cells (FIGS. 1 and 2). The DNA isolation robot comprises means for culturing a multiplicity of host cells, for example a block or microtiter plate. The culture volumes for the host cells are preferably in the range from 0.1-2.5 ml, particularly preferably in the range from 0.5 to 1 ml. Furthermore, the apparatus comprises means for obtaining plasmid DNA from a multiplicity of host cells, which means may be, for example, microtiter plates or blocks, which can, where appropriate, contain mini columns for purifying the plasmid DNA. The transfection robot contains means for transfecting and culturing a multiplicity of target cells, which can likewise be blocks or microtiter plates. Finally, the apparatus contains means for determining the activity of a reporter vector in target cells, preferably a spectrophotometric instrument or a fluorimetric instrument. Both robots employ multichannel pipettes, it being possible to pipette the particular liquids simultaneously in order to achieve an appropriate sample throughput.

A multiplicity of possible robot embodiments is possible in order to carry out screening. FIGS. 1 and 2 show two preferred embodiments in which the treated plates with the DNA samples and the host cells or the DNA samples and the target cells are laid out across an area and are linked to one another by a pipetting head which is movable in x, y, z direction.

The figures described below furthermore illustrate the invention in more detail.

FIG. 1 shows a diagrammatic representation of the DNA isolation robot. Storage places for blocks, for example 96-well blocks with host cells or DNA isolated therefrom and its intermediates, are denoted A, B, C, d. The reagents necessary for obtaining the DNA are arranged on both sides. P1, P2, P4, SiOx (silicon oxide solution), Acet. (acetone solution), and H₂O denote reservoirs with appropriate solutions required for obtaining the DNA. A washing station (“washing”) is required for cleaning the spray nozzles of the pipetting head. Means for centrifuging, (“centrifuge”), for applying reduced pressure (“vacuum”), for shaking (shaking) and incubation stations (inc.) serve to treat the plates for purifying the DNA. A pipetting head with claw arm, which can be moved in various directions across the whole area by drives (X, Y, Z), interconnects the plates and the treatment stations. The upper half of the illustration shows the robot in a side view.

FIG. 2 shows a diagrammatic representation of the transfection robot. The target cells for transfection are drawn in multiwell plates in four realms arranged above one another. The DNA samples to be transfected are arranged in 96-well plates in the topmost row. In the row below, the transfection reactions are mixed. The reagents required for this in addition to DNA are denoted L1 and L2. A washing station as well as a waste station (waste) serve to clean the spray nozzles of the pipetting head (“Z”) which is movable in X, Y, Z direction and driven by appropriate step motors (X, Y, Z). In the upper, right-hand edges of the illustration, side views of the robot and of the movable pipetting head, respectively, are drawn in.

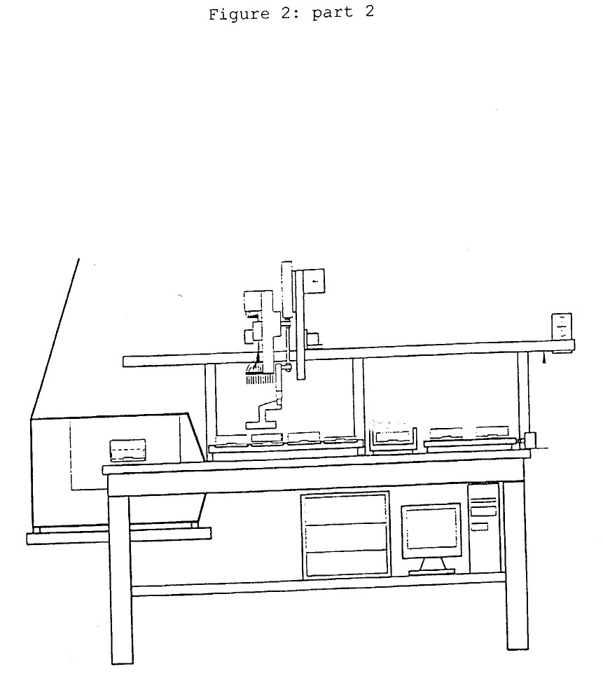

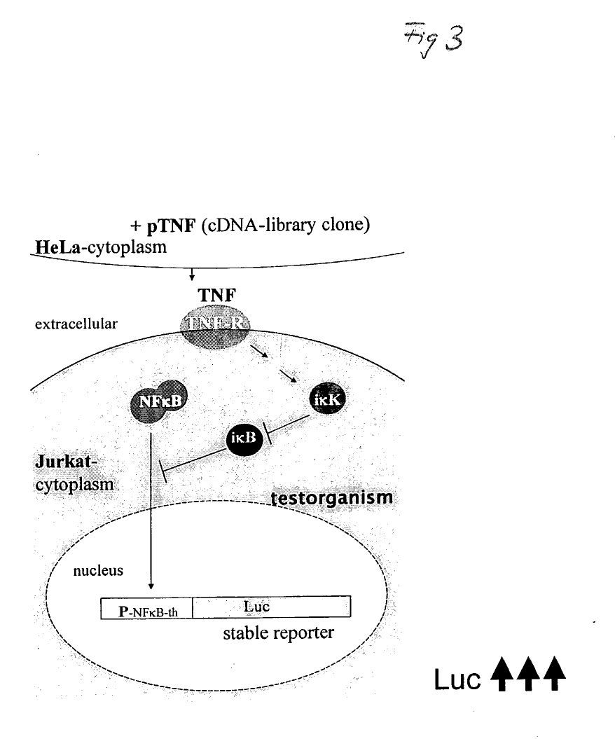

FIG. 3 shows a diagrammatic representation of the activation of the NFκB signal transduction pathway. According to the Use Example 1, the target organism (Hela cells) secretes TNF alpha which binds to a TNF receptor of a test organism which has been added to the screening mixture and has been transfected stably with the reporter gene. The signals then initiating from the TNF receptor activate a kinase complex of ikK kinases which in turn phosphorylate iκB and thus effect its release from the complex with NFκB. NFκB can then translocate into the nucleus where it can stimulate transcription of the NFκB-dependent gene for photinus pyralis luciferase.

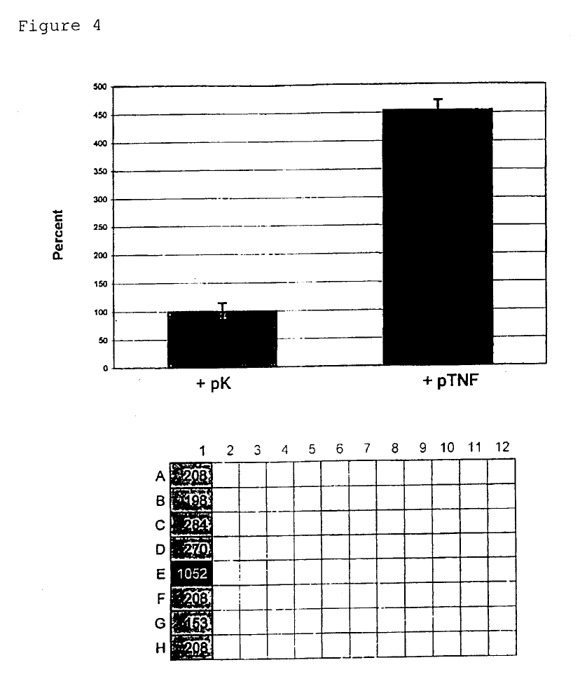

FIG. 4 shows the result of the screening for (secreted) factors acting on a test organism with the aid of the method of the invention. According to Use Example 1, the cDNA (plasmid pTNF) coding for the sequenced factor pTNF is detected by means of a test organism (JP4 cells) different than the transfected target organism (HeLa cells). Top part: average of relative luciferase activities (in percent). The 100% base value (gray bar) corresponds to the activity measured in transfection mixtures with the control plasmid (pcDNA3.1 (pcDNA 3.1=pK, Invitrogen). The black bar represents the average of the test organisms (JP4 cells) transfected with the test plasmid pTNF-alpha. The average is based in each case on at least three independent individual measurements. Bottom part: exemplary representation of individual measurements (in relative light units) in the wells of a microtiter plate, as typically generated during the screening. Wells marked in gray represent non-functional cDNAs in the target organisms (HeLa cells) with no change in reporter activity in the test organisms (JP4 cells). The well marked in black shows the effect of the functional cDNA (TNF-alpha) in the chosen read-out process.

FIG. 5 shows a diagrammatic representation of the inhibition of the NFκB signal transduction pathway by an inhibitory cDNA. According to the Use Example 2, JP4 cells are transfected prior to stimulation by recombinant TNF-alpha with a cDNA whose gene product, A20 protein, can negatively modulate signal transduction from the TNF receptor to NFκB (Song et al., PNAS 93 (1996), 6721-6725). NFκB activation is diminished, leading to inhibition of the transcription of the NFκB-dependent gene for photinus pyralis luciferase.

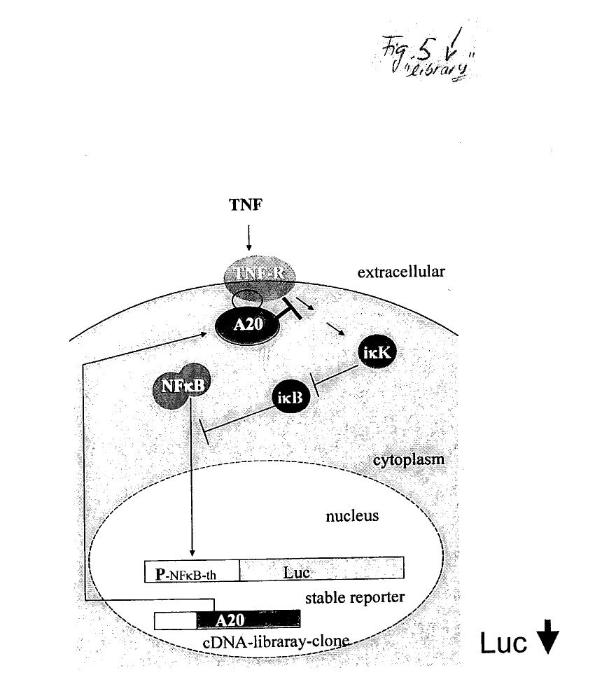

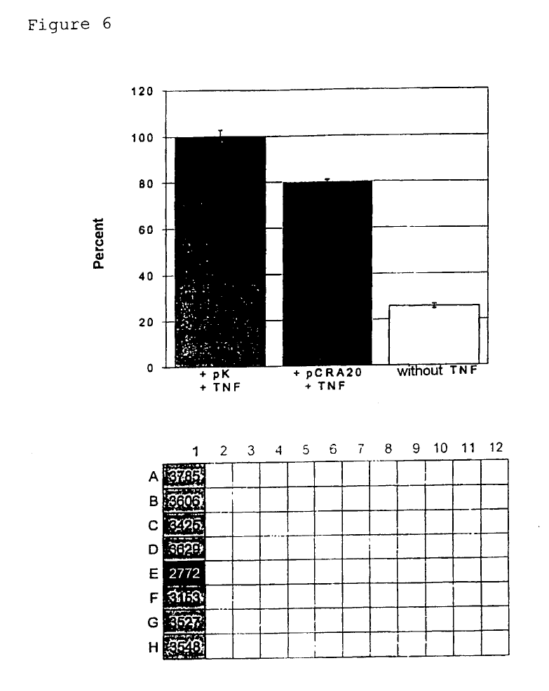

FIG. 6 shows the result of carrying out the method of the invention as a two-stage protocol. According to Use Example 2, an inhibitory cDNA (plasmid pCRA20) which blocks induction of a specific gene expression sequence (NFκB-dependent gene for photinus pyralis luciferase) is identified. Top part: average of relative luciferase activity (in percent). The 100% base value (gray bar) corresponds to the luciferase activity after stimulation with TNF-alpha, measured in transfection mixtures with the control plasmid (pcDNA3.1=pK, Invitrogen). The black bar represents the average of the target organisms (JP4 cells) transfected with the test plasmid pCRA20 and subsequent stimulation with TNF-alpha. The average is based on in each case at least three independent individual measurements. The white bar represents the base luciferase activity in JP4 cells not stimulated with TNF-alpha as a control. Bottom part: exemplary representation of individual measurements in the wells of microtiter plate, as they are typically generated during the screening. Wells marked in gray represent non-functional cDNAs (pcDNA3.1=pK, Invitrogen) which were linked with no change in reporter gene activity in the target organisms (JP4 cells). The well marked in black shows the effect of the functional cDNA (pCRA20) in the chosen read-out process.

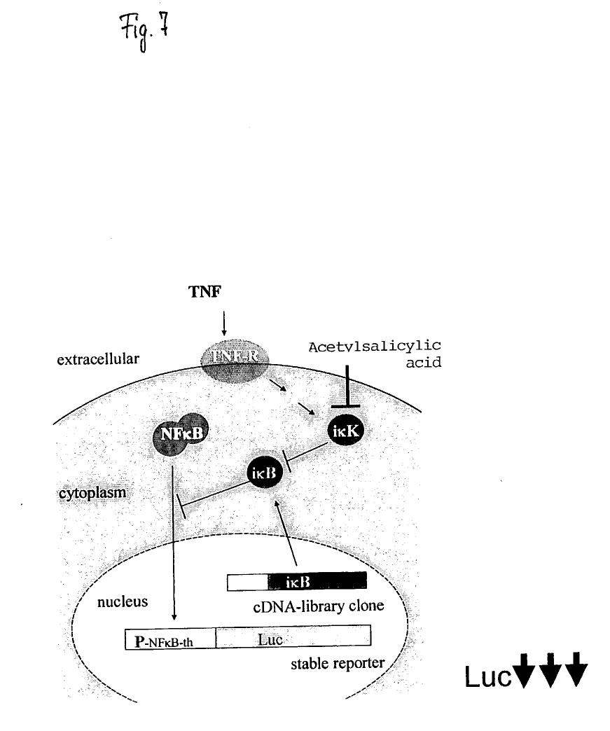

FIG. 7 shows the diagrammatic representation of an amplification of the action of a medicament, using aspirin as an example. According to the Use Example 3, addition of aspirin to the read-out system described leads in the target organisms (JP4 cells) to stabilization of iκB, since aspirin inhibits the activity of the iκB kinase complex which is responsible for degradation of iκB and thus for the release of NFκB. The result is a reduced activatability of the NFκB-dependent luciferase reporter gene by NFκB.

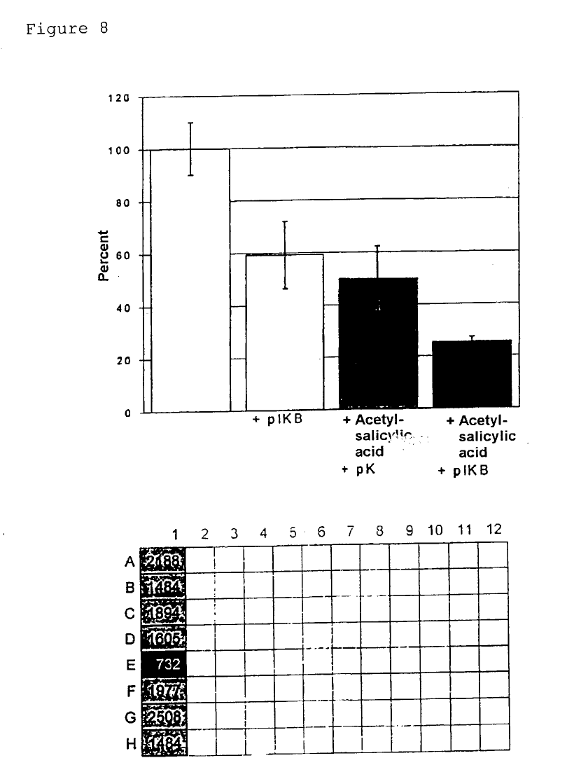

FIG. 8 shows the result of applying the method of the invention in order to determine genes which enhance the activity of pharmaceuticals. In Use Example 3 cotransfection of iκB enhances inhibition of the inducible transcription factor NF-κB by aspirin (acetylsalicylic acid). Top part: averages of relative luciferase activities (in percent). The 100% base value (white bar on the far left) corresponds to the luciferase activity in JP4 cell extracts after stimulation with TNF-alpha. Both the separate transfection of plasmid piκB and the addition of acetylsalicylic acid and control plasmid (pcDNA3.1=pk; Invitrogen) in each case partially reduce luciferase activity (white bar on the right and gray bar). The black bar represents the average of target organisms (JP4 cells) transfected with piκB and subsequent stimulation with TNF-alpha and incubation with acetylsalicylic acid. The average is based in each case on at least three independent individual measurements. Bottom part: exemplary representation of individual measurements in the wells of a microtiter plate, as are typically generated during the screening. Wells marked in gray represent non-functional cDNAs (pcDNA3.1=pK, Invitrogen) which had not been linked to any change in reporter activity in the target organisms (JP4 cells). The well marked in black shows the effect of the functional cDNA (plasmid piκB) in the chosen read-out process.

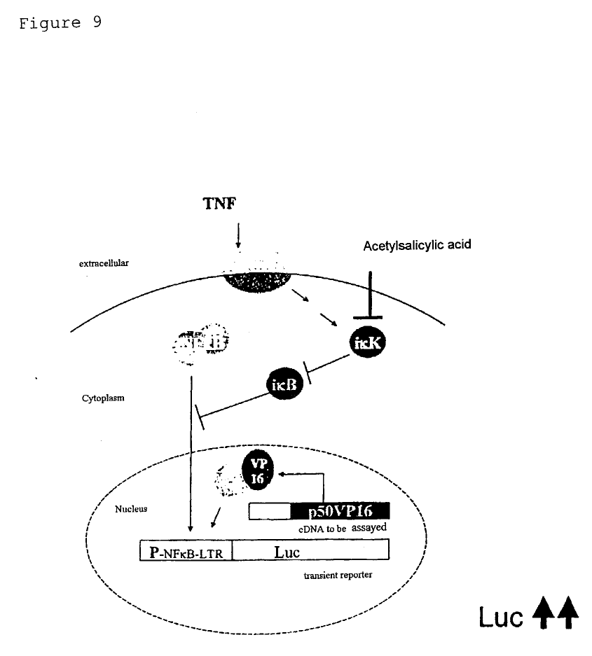

FIG. 9 shows the diagrammatic representation of the functional removal of the action of the medicament aspirin by the cDNA (p50VP16) of the p50 subunit of transcription factor NFκB, which is also fused to the VP16 transactivation domain. According to Use Example 4, expression of p50VP16 results in NFκB-activated expression of the luciferase reporter gene, since the site of p50VP16 action along the TNF-alpha signal chain is directly at the NFκB-dependent promoter downstream of the site of aspirin action.

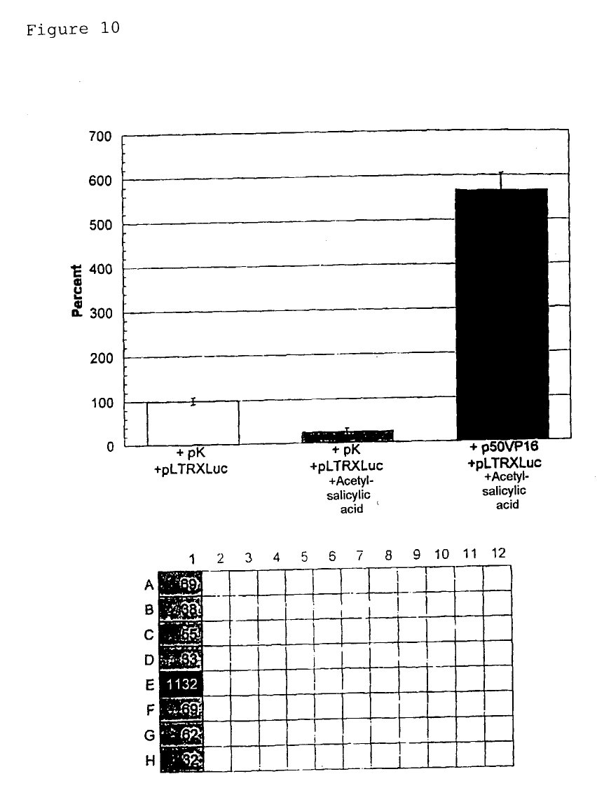

FIG. 10 shows the result of applying the method of the invention in order to determine genes which reduce the activity of pharmaceuticals. According to the Use Example 2, cotransfection of the cDNA (p50VP16) of the p50 subunit of transcription factor NFκB, to which is also fused the VP16 transactivation domain, removes the action of the medicament aspirin (acetylsalicylic acid). Top part: averages of relative luciferase activities (in percent). The 100% base value (white bar) corresponds to the luciferase activity in Jurkat cell extracts after stimulation with TNF-alpha. The gray bar represents reduction in luciferase activity when adding acetylsalicylic acid (and merely transfection of the control plasmid pcDNA3.1 (pcDNA3.1=pK, Invitrogen). The black bar represents the average of target organisms (Jurkat cells) transfected with the plasmid p50VP16 and subsequent stimulation with TNF-alpha and incubation with acetylsalicylic acid. The average is based in each case on at least three independent individual measurements. Bottom part: exemplary representation of individual measurements in the wells of a microtiter plate, as are typically generated during the screening. Wells marked in gray represent non-functional cDNAs (pcDNA3.1) which had not been linked to any change in reporter gene activity in the target organisms. The black well marked shows the effect of the functional cDNA (plasmid p50VP16) in the chosen read-out process.

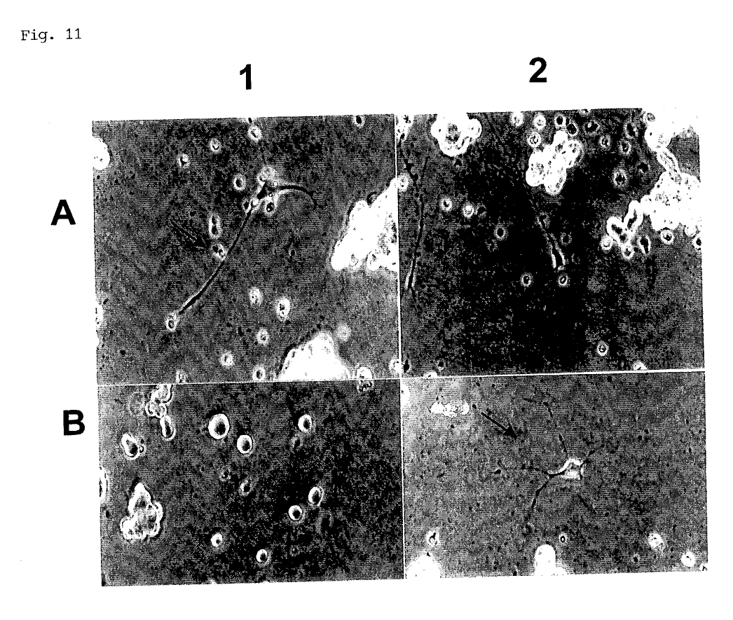

FIG. 11 shows the result of screening for the induction of a microscopically and optically detectable modification of cells with the aid of the method of the invention, using morphological differentiation as an example. According to Use Example 5, transfection of constitutively active ras protein (pV12ras) induces the formation of neurites in neuronal PC12 cells. pV12ras-transfected PC12 cells (A1 and A2) show distinct neurite formation (arrows) which is microscopically detectable. This morphology corresponds to control-transfected target organisms (B2) which were stimulated by NGF after transfection to form neurites. Only control-transfected cells (B1) showed in none of the experiments comparable structures of morphological differentiation.

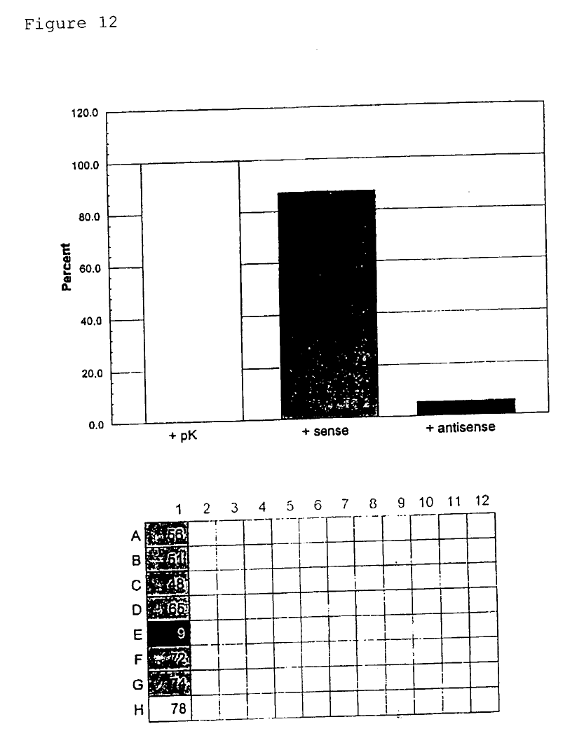

FIG. 12 shows the result of screening for translation-inhibiting (antisense) nucleic acids using the method of the invention. According to Use Example 6, an antisense oligonucleotide (as-oligo) against the transcript p50VP16 was transfected. Top part: averages of relative luciferase activities (in percent). The 100% base value (white bar) corresponds to the luciferase activity in cell extracts of Hela cells transfected with control plasmid after stimulation with TNF-alpha. The gray bar represents luciferase activity when adding sense oligonucleotide. The black bar represents the average of target organisms (Jurkat cells) transfected with the antisense oligonucleotide. The average is based in each case on at least three independent individual measurements. Bottom part: exemplary representation of individual measurements in the wells of a microtiter plate, as are typically generated during the screening. Wells marked in gray (or white) represent s-oligos (or control plasmid) which are linked only to a small (or no) change in reporter gene activity in the target organisms. The well marked in black shows the effect of the functional antisense oligos in the chosen read-out process.

The examples below are intended to illustrate the invention in more detail.

### EXAMPLES

The method of the invention comprises determination of the activity of the nucleic acid sequence in the individual populations of target organisms. If the target organism contains a reporter vector, it is possible to use changes in the activity of the reporter vector as a measure of the activity of the nucleic acid sequence to be studied. In Examples 1 to 4 and 6 below the gene product encoded by the reporter vector is the enzyme luciferase (Lui et al., Gene 202 (1977), 141-148). The report vector used were reporter plasmid constructs (Schwartz et al., Gene 88 (1990), 197-205) carrying the gene for photinus pyralis luciferase under the control of an NFκB-dependent promoter. Transcription factor NFκB is activated via a signal cascade whose starting point is tumor necrosis factor alpha (TNF-alpha). TNF-alpha is a secreted polypeptide which can bind to target cells having a TNF receptor. Binding to the receptor initiates the signal cascade in the target cells, leading to activation of NFκB. NFκB is present in the cytoplasm in inactive form, complexed with the inhibiting protein iκB. The signals coming from the TNF receptor activate a kinase complex of ikK kinases which in turn phosphorylate iκB and thus effect its release from the complex with NFκB. NFκB can now translocate into the nucleus of the cell where it is able to stimulate transcription of the NFκB-dependent gene for photinus pyralis luciferase.

### Example 1

Screening for (Secreted) Factors Acting on a Test Organism.

In an experimental setup corresponding to the present invention, it is intended to detect cDNAs coding for secreted factors. Detection is intended to be carried out by means of a test organism different than the transfected target organism. A significant increase in reporter gene activity in the test organisms is screened for.

The plasmid pTNF (pTNF=pcDNA3-delta-75-47, 32-1; Ishisaki et al., J. Biochem. 126 (1999), 413-420) which codes for the secreted tumor necrosis factor alpha (TNF-alpha) is transfected into the target organisms (Hela cells). As a control, the vector pcDNA3.1 (pcDNA3.1=pK, Invitrogen) which possess no functional cDNA in the present example is transfected in parallel into the target organisms. The test organisms are human T cells (Jurkat cells) which had been stably transfected beforehand with a reporter plasmid (clone JP4) which possesses a luciferase gene under the control of an NFκB-dependent promoter.

The Hela cells adherently growing in 96-well plates are transfected by lipofection. After 18 hours, the test organisms (JP4 cells) are added to the culture of the transiently transfected HeLa cells for 6 hours and then luciferase activity in the JP4 cell extract is determined by means of a luminescence reader. FIG. 3 shows diagrammatically the NFκB signal transduction pathway which causes the increased expression of the reporter enzyme luciferase.

The results of the use example are shown in FIG. 4. After transient transfection, the target organism secretes TNF-alpha which binds to the test organism (JP4 cells) which has been added to the screening mixture and has been stably transfected with a reporter plasmid. This results in activation of the NFκB signal transduction pathway in the test organism (JP4 cells) and this causes increased expression of the reporter enzyme luciferase.

This shows that it is possible to detect cDNAs coding for secreted factors in an experimental setup corresponding to the invention.

### Example 2

Two-Stage Protocol: Blocking the Induction of a Specific Gene Expression by an Inhibitory cDNA.

In an experimental setup corresponding to the present invention, the two-stage protocol uses human T cells (Jurkat cells) growing in suspension, which had been stably transfected beforehand with a reporter plasmid (clone JP4) which possesses a luciferase gene under the control of an NFκB-dependent promoter. Prior to stimulation with recombinant TNF-alpha, the JP4 cells are transfected with a cDNA (plasmid pCRA20) whose gene product (A20 protein) can negatively modulate signal transduction from the TNF receptor to NFκB (Song et al., PNAS 93 (1996), 6721-6725). As a control, the plasmid pcDNA3.1 (pcDNA3.1=pK, Invitrogen) which, in the present example, carries no cDNA is transfected.

1st stage: JP4 cells are transiently transfected in microtiter plates by means of lipofection.

2nd stage: After 24 hours, stimulation with TNF-alpha is carried out for 2.5 hours. This is followed by screening for a significant decrease in reporter gene activity in the target organisms (JP4 cells) in which luciferase activity is determined in the cell extract by means of a luminescence reader. FIG. 5 shows diagrammatically the signal transduction from NF receptor to NFκB, which is negatively modulated by the cDNA gene product (A20 protein).

The results of the use example are shown in FIG. 6. After transient transfection, the target organism (JP4 cells) secretes A20 protein which blocks signal transduction of the TNF receptor bound to TNF-alpha. NFκB activation is reduced and, as a result, transcription of the NFκB-dependent reporter gene is inhibited. This results in a significant reduction in luciferase activity in the target organisms. This shows that it is possible to detect inhibitory cDNAs which block induction of a specific gene sequence in an experimental setup according to the invention.

### Example 3

Enhancing the Action of a Medicament

It is possible with the aid of the method of the invention to determine genes which can enhance the activity of pharmaceuticals. Acetylsalicylic acid (tradename Aspirin) inhibits the activity of the iκK kinase complex (Kwak et al., J. Biol. Chem. 275 (2000), 14752-14759) which is responsible for iκB degradation (Karin, M., Oncogene 18 (1999), 6867-6874).

The iκB cDNA (plasmid piκB) is transiently transfected into the target organisms (human T cells (Jurkat cells which have been stably transfected beforehand with a reporter plasmid which possesses the photinus pyralis luciferase gene under the control of an NFκB-dependent promoter (JP4 cells)). The plasmid pcDNA3.1 (pcDNA3.1=pK, Invitrogen) which possesses no functional cDNA in the present example serves as a control.

JP4 cells were transfected in microtiter plates by means of lipofection. After incubating for 20 hours, JP4 cells were stimulated with TNF-alpha and simultaneously treated with an acetylsalicylic acid dosage. The cells are screened for a significant increase in the partial inhibition of the TNF-alpha-dependent luciferase activity by determining luciferase activity in the cell extract by means of a luminescence reader. FIG. 7 shows diagrammatically the action of the medicament acetylsalicylic acid on the selected target organism.

The results of the use example are shown in FIG. 8 which clearly shows that the addition of aspirin to the read-out system described in the target organisms (JP4 cells) stabilizes iκB and thus reduces in a concentration-dependent manner the activatability of the luciferase reporter gene by NFκB (partial reduction in TNF-alpha-dependent luciferase activity in the cell extract).

### Example 4

Functional Removal of the Action of a Medicament

The method of the invention can be employed in order to determine genes which can inhibit or extinguish the activity of pharmaceuticals. This use example is intended to demonstrate the removal of the action of the medicament acetylsalicylic acid (trade name Aspirin) by means of a suitable cDNA (p50VP16). Acetylsalicylic acid inhibits the activity of the iκK kinase complex (Kwak et al., J. Biol. Chem. 275 (2000), 14752-14759), which is responsible for iκB degradation (Karin, M., Oncogene 18 (1999), 6867-6874). A suitable cDNA in this example is the cDNA (p50VP16) of the p50 subunit of transcription vector NFκB (NFκB is a dimer composed of a p50 and a p65 subunit (Schmitz and Bäuerle, EMBO 10 (1991), 3805-3817)) to which had also been fused the VP16 transactivation domain. The site of p50VP16 action is located directly at the NFκB-dependent promoter downstream of the site of acetylsalicylic acid action, along the TNF-alpha signal chain.

The plasmid p50VP16 is transiently transfected into the target organisms (Jurkat cells). Additionally, the luciferase reporter plasmid pLTRXLuc is transiently cotransfected into the target cells. The plasmid pcDNA3.1 (pcDNA3.1=pK, Invitrogen) which possessed no functional cDNA serves as a control.

Jurkat cells were transfected with the plasmids p50VP16 and pLTRXLuc in microtiter plates by means of lipofection. After incubating for 22 hours, the target cells are stimulated with TNF-alpha and simultaneously treated with a dosage of acetylsalicylic acid. The target cells are screened for a significant increase in luciferase activity by determining the luciferase activity in the cell extract by means of a luminescence reader. FIG. 9 shows diagrammatically the action of the medicament acetylsalicylic acid on the target organism.

The results of the use example are shown in FIG. 10 and they show that the addition of aspirin to the readout system described in the target organisms (Jurkat cells) leads to a concentration-dependent increased activatability of the luciferase reporter gene by NFκB. The cDNA of the p50 subunit of transcription factor NFκB, to which had also been fused the VP16 transactivation domain, therefore causes functional removal of the action of the medicament acetylsalicylic acid, since the site of p50VP16 action is located along the TNF-alpha signal curve downstream of the site of aspirin action.

### Example 5

Screening for the Induction of a Microscopically and Optically Detectable Modification of Cells, Using the Example of a Morphological Differentiation

The biological activity of the nucleic acid sequence can be determined in principle by determining any phenotypically recognizable effects such as, for example, morphological modifications which are caused by transfection of the nucleic acid into the target organism.

PC12 cells are cultivated on collagen and transfected with constitutively active ras protein (pV12ras) (FIG. 5, A1 and A2). Transfection of the plasmid pcDNA3.1 (pcDNA3.1=pK, Invitrogen; FIG. 5, B1 and B2), which has no functional cDNA serves as a control. The target cells are subsequently incubated in differentiation medium for 48 hours. 24 hours after transfection, one part of the cells transfected with the control plasmid pcDNA3.1 (FIG. 11, B2) is stimulated with NGF to form neurites.

The results of the use example are shown in FIG. 11 and clearly show that transfection of constitutively active ras protein (pV12ras) into neuronal PC12 cells induce the formation of neurites.

### Example 6

Screening for Translation-Inhibiting (Antisense) Nucleic Acids

The nucleic acid sequences to be studied using the method of the invention can in principle come from any sources and may be, for example, genomic sequences, cDNA sequences, cDNA fragments or synthetically generated sequences such as, for example, antisense molecules or combinatorily modified nucleic acid sequences of any origin. In this use example, it is intended to screen for the activity of a translation-inhibiting (antisense) nucleic acid.

Hela cells are transiently transfected with the luciferase reporter plasmid pLTRXLuc, plasmid p50VP16 (cDNA of the p50 subunit of transcription factor NFκB, to which is also fused the VP16 transactivation domain) and either antisense oligonucleotide (as-oligo, corresponds to NFκB1 according to Reuning et al., NAR 23 (1995), 3887-3893) or sense oligonucleotide or the control plasmid pcDNA3.1 (pcDNA3.1=kP, Invitrogen) The antisense oligonucleotide (as-oligo) acts against the p50VP16 transcript. After incubating the Hela cells in microtiter plates for 36 hours, luciferase activity in the cell extract was analyzed.

The results of the use example are shown in FIG. 12. They clearly show that the antisense oligonucleotide (as-oligo) reduces reporter gene activity on the cotransfected reporter plasmid pLTRXLuc (Schmitz and Bäuerle, EMBO 10 (1991), 3805-3817). With the aid of the screening procedure of the invention it was possible to clearly detect the activity of the translation-inhibiting (antisense) nucleic acid.

### Example 7

Read-Out for Cell Cycle Regulation/-Inhibition

In order to isolate from a cDNA library genes whose overexpression has a specific influence on the cell cycle of the target cells, said cells are transiently transfected on a 96-well scale with a cDNA library described in the description. After incubation for 48 hours, the cells are permeabilized using a method known to the skilled worker (e.g. using Nonidet P-40) and, after addition of a DNA-binding fluorescent dye (e.g. propidium iodide), analyzed for DNA content in a flow cytometer. If a cDNA codes for a gene which makes the cells lock in a section of the cell cycle, then this is readily detectable in FACS analysis by the skilled worker.

Overexpression of the protein p21 gene, for example, shows an effect of this kind for a G1 phase arrest; this has been described in the relevant literature, for example by Gartel et al., Proc. Soc. Exp. Biol. Med. 213 (1996), 138-149.

### Example 8

Virus-Infected Cells

According to the method of the invention, it is possible to infect the target organisms before, during or after introducing the nucleic acid sequence to be studied with viruses and to determine the influence of the nucleic acid sequence to be studied on the behavior of the target organisms toward the viruses, for example inhibition or activation of viral replication or penetration of the virus into the cells.

In order to isolate from a cDNA library genes whose overexpression specifically causes or, in another embodiment, prevents apoptosis of virus-infected cells, the virus-infected cells are transiently transfected on the 96-well scale with a cDNA library described in the description. After appropriate incubation of the virus-infected cells, the apoptosis read-out described detects whether a cDNA codes for a gene which drives the virus-infected cell into apoptosis or, in the second embodiment, prevents just that.

For example, the E1A virus protein drives an affected cell into apoptosis. If however, the E1A-containing cell is transfected with the apoptosis suppressor bcl-2 and if said suppressor is overexpressed, the E1A-affected cell does not go into apoptosis (Rao et al., PNAS 89 (1992), 7742-7746). Here, transfection of the cells with the E1A mimics infection with the virus.

### Example 9

Functional Complementation

If patient cells which have a genetic defect are used as target organisms according to the method of the invention, it is possible to search for nucleic acid sequences which are capable of repairing said defect.

In order to isolate from a cDNA library genes whose overexpression complements the cellular phenotype of a (genetic) disorder, characterized patient cells are transiently transfected with a cDNA library according to the description of the invention of a healthy donor. After appropriate incubation, the cells are screened for the cellular phenotype in the read-out by means of methods known to the skilled worker and analyzed for complementation of the defect.

Cells of a patient of Fanconi anemia complementation group C (HSC536), which contain mutations in the responsible Fanc-C gene, are transfected, for example, with the wild type Fanc-C gene. When studying the cellular phenotype (mytomycin C oversensitivity for proliferation and G2-phase arrest in the cell cycle), correction of said phenotype by the wild type gene can be detected (e.g. Machl et al., Gene Therapy 4 (1997), 339-345). Thus, using the method of the invention, it is possible to identify genes connected to diseases much more readily than using conventional methods (such as, for example, positional cloning).

1. A method for determining the biological activity of nucleic acid sequences, comprising the steps:
(a) parallel introduction of a multiplicity of expression vectors containing in each case a nucleic acid sequence to be studied operatively linked to an expression control sequence into a multiplicity of in each case separate populations of target organisms of the same type, where in each case only a single nucleic acid sequence or a small number of various nucleic acid sequences are introduced into a separate population of target organisms of the same type,
(b) effecting of expression of the nucleic acid sequence in the target organisms, and
(c) determination of the activity of the nucleic acid sequence in the individual populations of target organisms.

2. The method as claimed in claim 1,
characterized in that
the nucleic acid sequences are selected from among genomic sequences, cDNA sequences, cDNA fragments and antisense molecules of any origin.

3. The method as claimed in claim 1 or 2,
characterized in that
nucleic acids suitable for RNAi are used.

4. The method as claimed in any of claims 1 to 3,
characterized in that
the nucleic acid sequences come from a normalized cDNA library.

5. The method as claimed in any of claims 1 to 3,
characterized in that
the nucleic acid sequences come from libraries containing a collection of known sequences.

6. The method as claimed in any of claims 1 to 5,
characterized in that
the nucleic acid sequences come from eukaryotes, bacteria, archaebacteria, viruses or from synthetic or semisynthetic sources.

7. The method as claimed in any of claims 1 to 6,
characterized in that
the nucleic acid sequences have a non-selectable activity.

8. The method as claimed in any of claims 1 to 6,
characterized in that
the nucleic acid sequences have a selectable activity.

9. The method as claimed in any of the preceding claims,
characterized in that
the expression vector used is a plasmid.

10. The method as claimed in any of claims 1 to 9,
characterized in that
the expression vector is provided by culturing host cells and obtaining said vector from the cultured host cells.

11. The method as claimed in any of claims 1 to 9,
characterized in that
the expression vector is provided by an in-vitro amplification.

12. The method as claimed in any of the preceding claims,
characterized in that
the target organisms used are, where appropriate, genetically manipulated eukaryotic cells or organisms, where appropriate genetically manipulated prokaryotic cells, cells of patients or natural mutants.

13. The method as claimed in claim 12,
characterized in that
the target organisms used are eukaryotic cells and the expression vector is introduced by calcium phosphate coprecipitation, lipofection, electroporation, particle bombardment or viral infection.

14. The method as claimed in any of the preceding claims,
characterized in that
the expression vector is introduced into the target organisms together with a reporter vector.

15. The method as claimed in any of the preceding claims,
characterized in that
the expression vector is introduced into target organisms which already contain a reporter vector.

16. The method as claimed in claim 14 or 15,
characterized in that
the reporter vector is located at a specific site in the genome.

17. The method as claimed in any of claims 14 to 16,
characterized in that
the reporter vector contains an expressible nucleic acid sequence coding for a detectable gene product.

18. The method as claimed in claim 17,
characterized in that
the detectable gene product is selected from secreted enzymes.

19. The method as claimed in claim 17,
characterized in that
the detectable gene product is selected from intracellulary detectable polypeptides.

20. The method as claimed in claim 17 or 18,
characterized in that
the detectable gene product is selected from fluorescent proteins.

21. The method as claimed in claim 17,
characterized in that
the detectable gene product is selected from membrane-bound detectable polypeptides.

22. The method as claimed in any of the preceding claims,
characterized in that
determination of the activity of the nucleic acid sequence comprises studying the target organisms for morphological changes, changes in growth behavior, changes in the cell cycle or changes in the activity of a reporter vector.

23. The method as claimed in any of claims 1 to 22,
characterized in that
additional parameters of the target organisms are analyzed.

24. The method as claimed in any of claims 1 to 23,
characterized in that
the determination is carried out by fluorescence cytometry or imaging assays.

25. The method as claimed in any of the preceding claims,
characterized in that
the procedure is at least partly automated.

26. The method as claimed in any of the preceding claims,
characterized in that
steps (a) to (c) are in each case carried out in parallel for at least 50 populations of target organisms.

27. The method as claimed in any of the preceding claims, comprising the addition of a stimulus whose activity can be influenced in the target organism by the nucleic acid to be studied.

28. The method as claimed in claim 27,
characterized in that
the stimulus is generated by a pharmacon.

29. The method as claimed in any of the preceding claims, comprising the addition in step (b) of a test organism which is different from the target organism.

30. The method as claimed in claim 29,
characterized in that
the test organism used is a cell line.

31. The method as claimed in claim 29,
characterized in that
the test organism used is a virus or bacterium.

32. The method as claimed in any of claims 29 to 31,
characterized in that
the action of the nucleic acid sequence to be studied on the test organism is determined in step (c).

33. The method as claimed in any of the preceding claims for optimizing nucleic acid sequences.

34. The method as claimed in claim 33,
characterized in that
a multiplicity of variants of a known nucleic acid sequence is assayed.

35. The method as claimed in any of the preceding claims, furthermore comprising the use of the identified nucleic acid sequences for providing diagnostic and therapeutic agents.
